(19) United States
(12) Patent Application Publication
(10) Pub. No.: US 2015/0104348 A1
(43) Pub. Date: Apr. 16, 2015

(54) Pet Deodorizing Composition

(71) Applicant: The Iams Company, Cincinnati, OH (US)

(72) Inventors: Ryan Matthew Nichols, Cincinnati, OH (US); Emma Lynne Klingman, Wyoming, OH (US); John Brian Strube, Okeana, OH (US)

(73) Assignee: The Iams Company, Cincinnati, OH (US)

(21) Appl. No.: 14/050,550

(22) Filed: Oct. 10, 2013

(51) Int. Cl. A61K 8/73; A61Q 5/02; A61K 8/362; A61Q 15/00; A01K 13/00; A61K 8/58

(52) U.S. Cl. CPC A61K 8/738; A01K 13/00; A61K 8/585; A61K 8/362; A61Q 15/00; A61Q 5/02
USPC 422/5; 424/70.21

(57) ABSTRACT

A deodorizing composition that entraps and neutralizes malodors and is intended to be used on a pet. The deodorizing composition contains cyclodextrin, an aqueous carrier, a water soluble silicone, and a buffer system comprising citric acid. The composition has a pH from about 5 to about 8.5 and the composition is non-irritating and non-toxic to pets.

### FIELD OF THE INVENTION

The present invention is directed towards a deodorizing composition intended for use on pets.

### BACKGROUND OF THE INVENTION

People tend to spend a lot of time with their pets, especially pets that live inside their homes. However, many pets develop a malodor. Malodor can be caused by aerobic and anaerobic bacteria and yeast that degrade body sebum, oils, and saliva that are naturally deposited on a pet's coat. Pets' coats can also entrap soil, leaves, grass, and animal discharge, which can decompose and further contribute to the pets' malodor.

One way to remove pet odor is to give the pet a bath. However, frequent bathing can make a pet's coat dry and dull, stripping the coat of its natural sheen and water resistance, and can irritate a pet's sensitive skin. Furthermore, bathing a pet can be time consuming and difficult, especially if the pet is not fond of baths.

Thus, many pet owners use pet deodorizing products to cover up malodor. One problem with many of these products is that they do not neutralize malodor, instead they simply cover up a pet's odor with perfume. Other sprays can irritate a pet's sensitive skin if applied directly to a pet's coat or applied using a cleaning implement such as a rag or paper towel. Furthermore, since many pets self-groom, they can ingest some of the deodorizing spray, thus a spray that is not formulated especially for pets can lead to an upset stomach and could even be toxic. Furthermore, traditional deodorizing sprays do not provide a mechanism for removing odor causing agents from a pet's coat.

Thus, there is a need for a non-irritating, non-toxic, deodorizing spray that removes malodors that can be applied to a pet.

### SUMMARY OF THE INVENTION

A deodorizing composition that entraps and neutralizes malodors on a hair covered surface of a mammal comprising: a cyclodextrin; an aqueous carrier; a water soluble silicone with an HLB value that is greater than about 6; and a buffer system comprising citric acid; wherein the deodorizing composition has a pH from about 5 to about 8.5 and wherein the deodorizing composition is non-irritating and non-toxic to mammals.

A deodorizing composition that entraps and neutralizes malodors on a pet comprising: a cyclodextrin; an aqueous carrier; and a perfume; wherein the deodorizing composition is non-irritating and non-toxic to pets and wherein the deodorizing composition is adapted to be used with a dirt lifter comprising a front side comprising textured portions wherein the dirt lifter is adapted to distribute the deodorizing composition through a pet's coat.

A method of entrapping and neutralizing malodors on a pet comprising: (a) applying a deodorizing composition on a pet wherein the deodorizing composition comprises a cyclodextrin, an aqueous carrier, and a perfume; (b) distributing the deodorizing composition across at least a portion of the pet's hair using a dirt lifting glove comprising a front side with textured portions and a back side wherein the front side is joined to at least a portion of the back side along the perimeter; wherein the deodorizing composition is non-toxic and non-irritating to a pet.

### BRIEF DESCRIPTION OF THE DRAWINGS

FIG. 1 is a photograph of a dirt lifter before it has been used;

FIG. 2 is a photograph of a dirt lifter after it has been used to clean and deodorize a pet;

FIG. 3 is a front view of a dirt lifter;

FIG. 4 is a back view of a dirt lifter shown in FIG. 3; and

FIG. 5 is a cross-section view of section 3-3 of FIG. 3.

### DETAILED DESCRIPTION OF THE INVENTION

Pets naturally develop malodor that can be unpleasant to human owners, especially when the pets are kept inside the home. For instance, dogs naturally produce scented secretions including musty secretions from two anal sacs. These secretions can help dogs and other pets identify one another, but they may not be pleasing to their human owners. Additionally, body sebum and other oils house bacteria and yeast which oxidize and the degrading oils have an unpleasant odor. Pets also self-groom leaving saliva and food debris on their coat, which can smell as it is degraded by anaerobic bacteria. Furthermore, a pet's coat, especially pets with long, thick, or corded hair, can trap soil, leaves, grass, and animal discharge that can have an unpleasant odor especially if this debris is left on the animal and decomposes. The pet odor problem is further exacerbated because many pets spend a significant amount of time outside, exposed to the elements, thus picking up more dirt and unpleasant odors that get trapped in their coats.

In order to get rid of the malodor, a pet can be bathed. However, bathing a pet can be burdensome and frequent bathing can irritate some pets' sensitive skin. Dogs, in particular have very sensitive skin. For instance, the stratum correum for human skin is 10 to 15 layers of cells thick, while the stratum corrneum for a dog is only 3 to 5 layers of cells thick. Thus, many pet owners bathe their pet infrequently, for instance once every four to six weeks.

There are currently products that are intended to help reduce pet malodor. However, many of these products simply conceal malodor, instead of neutralizing, entrapping, and/or removing it. Furthermore, if a deodorizing product is used and it is not specially designed for a pet it can irritate a pet's sensitive skin and in some instances it can be toxic.

Formulating a deodorizing composition that effectively neutralizes and encapsulates pet malodors and is non-irritating and non-toxic can be difficult, especially when considering the great variation among pets. It can be particularly difficult to formulate a composition that is non-toxic because many pets self-groom and may ingest some of the deodorizing composition. Also, some pets are very small weighing only a couple of pounds, and therefore ingesting what seems like a small amount, such as a few licks, can cause detrimental effects.

Furthermore, there is great variation among pets' coats. For instance, some pets are double coated and have a soft undercoat and a coarser topcoat. Other pets, including certain breeds of dogs such as the Chinese Crested and the Xoloitzcuintli, are essentially hairless. The texture of the coat can also vary and pet hair can be wiry, curly, smooth, coarse, fine, wavy, and combinations thereof. Some dogs, such as Pulis, Poodles, and Komondors, can have curly coats that are corded. The length of the coat also varies from short to vary long. Some pets have a coat that grows to a specific length and then stops growing and other pets have a coat that continuously grows. Furthermore, many pets shed and almost all pets lose hair and the old hairs are replaced with new ones. In one example, the deodorizing composition can be used on all pets. In another example, the deodorizing composition can be used on all breeds of dogs and in another example, the deodorizing composition can be used on all breeds of dogs except for hairless dogs. In another example, the deodorizing composition can be customized for the specific kind of coat that the pet has.

The pet deodorizing composition of the present invention can comprise water, ethanol, cyclodextrin, and perfume. The deodorizing composition can neutralize and entrap malodor without stripping the coat of its natural sheen and water resistance and without irritating the pet's sensitive skin. In one example, the composition further comprises an ingredient that provides conditioning benefits, such as a water soluble silicone. The deodorizing composition can be placed into a spray bottle for easy application either directly onto a pet or onto a separate implement, such as a dirt lifter, and then applied to the pet. The dirt lifter can help distribute the deodorizing spray and it can also help remove odor causing agents and malodor. In one example, the composition can be suitable for ingestion by a pet.

In some examples, the deodorizing composition can be used on human hair. Human hair can develop malodors from sweat, oil and environmental odors such as smoke, cooking odors, and pollution. A person may not want to wash her hair because washing hair can strip the hair of color, hair treatments such as perms or straightening treatments, and/or natural oils and it can be time consuming. Also, after washing hair some people expose their hair to harsh heat from a blow dryer, curling iron, or flat iron, which can damage hair.

In one example, the deodorizing composition is clear. The term “clear” as defined herein means transparent or translucent, preferably transparent as in “water clear,” when observed through a layer having a thickness of less than about 10 cm.

As used herein, “glove” refers to a covering for the hand having separate sections for each finger.

As used herein, “mitt” refers to a covering for the hand that is partially or wholly unseparated and that may include space for the thumb in the main enclosure or may provide space for the thumb in a separate enclosure for the thumb or may not include a thumb enclosure at all. This term is also applicable to an implement that covers only one or more digits of a user, such as in the case of a “finger mitt”.

While the terms “glove” and “mitt” have been defined with respect to the human hand, similar structures could be utilized to cover or enclose other elements of human anatomy, such as foot coverings, or other items for which coverings of a particular shape are preferred.

As used herein, the term “large pet” refers to a pet that is greater than or equal to 50 lbs. (22.7 kg).

As used herein, the term “medium pet” refers to a pet that is greater than 20 lbs. (9.1 kg) and less than 50 lbs. (22.7 kg).

As used herein, “odor causing agents” refers to anything that is causing malodor on a pet. Common sources of malodor can include body sebum and other oils, saliva, food debris, soil, leaves, grass, and animal discharge.

As used herein, “on-the-hand implement” refers to a covering for the hand or portion of the hand such as a finger, thumb, or palm.

As used herein, “pet” means dogs, cats, small mammals such as gerbils, hamsters, chinchillas, rats, rabbits, guinea pigs, and mustelids including ferrets and skunks, and/or other domesticated animals.

As used herein, the term “small pet” refers to a pet that is less than or equal to 20 lbs. (9.1 kg).

As used herein, “tuft”, refers to a particular type of protrusion that may be formed in a nonwoven web. Tufts typically have a tunnel-like configuration, and in some cases may be open at one or both of their ends.

As used herein, the articles “a” and “an” when used herein, for example, “an aqueous carrier” or “a deodorizing composition” is understood to mean one or more of the material that is claimed or described.

### Deodorizing Composition

Pets have sensitive skin and all of the ingredients and each ingredient level in the deodorizing composition must be carefully selected to be ingestible by a pet, to prevent irritation to a pet's skin, and to maintain a glossy healthy coat while neutralizing and entrapping malodor.

### Cyclodextrin

The deodorizing composition can comprise cyclodextrin. For reducing malodor impression on pets, while still being non-irritating and non-toxic to pets, the cyclodextrin can be used in low levels and still be effective. In one example, the composition comprises from about 0.1% to about 5% cyclodextrin, by weight of the composition, in another example from about 0.5% to about 4%, in another example from 1% to about 3%, in another example from about 1.5% to about 2.5%, and in another example from about 1.9% to about 2.3%. In one example, the composition comprises about 2.25% cyclodextrin.

In one example, the cyclodextrin is solubilized, water-soluble, and uncomplexed. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

In one example, the cyclodextrins used in the present invention are highly water-soluble such as, alpha-cyclodextrin and derivatives thereof, gamma-cyclodextrin and derivatives thereof, derivatised beta-cyclodextrins, and/or mixtures thereof. The derivatives of cyclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R is a methyl or an ethyl group; those with hydroxyalkyl substituted groups, such as hydroxypropyl cyclodextrins and/or hydroxyethyl cyclodextrins, wherein R is a —CH₂—CH(OH)—CH₃ or a —CH₂CH₂—OH group; branched cyclodextrins such as maltose-bonded cyclodextrins; cationic cyclodextrins such as those containing 2-hydroxy-3(dimethylamino)propyl ether, wherein R is CH₂—CH(OH)—CH₂—N(CH₃)₂ which is cationic at low pH; quaternary ammonium, e.g., 2-hydroxy-3-(trimethylammonio)propyl ether chloride groups, wherein R is CH₂—CH(OH)—CH₂—N⁺(CH₃)₃Cl⁻; anionic cyclodextrins such as carboxymethyl cyclodextrins, cyclodextrin sulfates, and cyclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., the mono-3-6-anhydrocyclodextrins, as disclosed in “Optimal Performances with Minimal Chemical Modification of Cyclodextrins”, F. Diedaini-Pilard and B. Perly, The 7th International Cyclodextrin Symposium Abstracts, April 1994, p. 49; and mixtures thereof. Other examples of cyclodextrin derivatives are disclosed in U.S. Pat. Nos. 3,426,011, 3,453,257, 3,453,258, 3,453,259, and 3,453,260, 3,459,731, 3,553,191, 3,565,887, 4,535,152, 4,616,008, 4,678,598, 4,638,058, and 4,746,734.

Highly water-soluble cyclodextrins are those having water solubility of at least about 10 g in 100 ml of water at room temperature, in another example at least about 20 g in 100 ml of water, and in another example at least about 25 g in 100 ml of water at room temperature. Examples of water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. In one example, the cyclodextrin is hydroxypropyl beta-cyclodextrin. The cyclodextrins are commercially available, e.g., from Wacker Chemie AG (Adrian, Mich.).

In one example, a mixture of cyclodextrins is used. Such mixtures can complex with a wider range of perfume molecules having a wider range of molecular sizes. In one example, at least a portion of the cyclodextrins is alpha-cyclodextrin and its derivatives thereof, gamma-cyclodextrin and its derivatives thereof, and/or derivatised beta-cyclodextrin, and mixtures thereof.

In one example, concentrated compositions can also be used in order to provide a less expensive product. When a concentration is used, i.e., when the level of cyclodextrin used is greater than about 3%, it is preferable to dilute the composition before putting the composition on the pet, in order to avoid irritation and possibly staining the pet's coat. In one example, the cyclodextrin is diluted with about 50% to about 2000%, by weight of the composition, water, in another example about from 60% to about 1000%, and in another example from about 75% to about 500%.

### Perfume

The deodorizing composition can comprise perfume. The perfume that can be selected for use in the deodorizing spray contains ingredients with odor characteristics which are preferred in order to provide a fresh impression, where the consumer can smell the perfume, on a pet's fur. In some examples, consumers experience a noticeable fresh impression on their pet for an extended period of time. In one example an extended period of time is greater than 1 hour, in another example greater than about 6 hours, in another example greater than about 12 hours, in another greater than about 24 hours, and in another example greater than about 36 hours. In another example, consumers experience a noticeable fresh impression for about 24 to about 48 hours. In another example, the consumer would prefer not to smell the perfume and the perfume smell dissipates after about 15 minutes. In another example, the composition does not comprise a perfume and the composition entraps and eliminates odors without leaving a noticeable scent.

In one example, the perfume only comprises one fragrance material. In another example, the perfume has more than one fragrance material. In one example, the first fragrance material can be initially perceived by the consumer when the composition is first applied to the pet and the second fragrance material can be perceived after the initial application.

In one example, the perfume can be encapsulated in the cyclodextrin and when the malodor is encapsulated by the cyclodextrin, the perfume will be released and the consumer will experience a fragrance bloom.

In another example, the perfume is encapsulated in microcapsules. The microcapsules will be activated when they are manipulated. The microcapsules can be manipulated by the user brushing or petting the pet when the pet makes certain movements, such as rolling around. Additional information on perfume microcapsules can be found in U.S. patent application Ser. Nos. 12/777,301, 12/777,304, 12/777,314, 12/777,318, 12/562,578, 12/064,506, and 12/328,340.

In some examples, the consumer may want to perceive a strong scent immediately after administering the deodorizing composition on their pet. This is especially true if the pet is really smelly, for instance a wet dog. A perfume that has a strong initial scent can be made by selecting perfume ingredients, including fragrance materials, which are not too hydrophobic. The less hydrophobic perfume ingredients are more soluble in water, and are more available in the deodorizing composition. The degree of hydrophobicity of a perfume ingredient can be correlated with its octanol/water partitioning coefficient P. The octanol/water partitioning coefficient of a perfume ingredient is the ratio between its equilibrium concentration in octanol and in water. A perfume ingredient with a greater partitioning coefficient P is more hydrophobic. Conversely, a perfume ingredient with a smaller partitioning coefficient P is more hydrophilic. The preferred perfume ingredients of this invention have an octanol/water partitioning coefficient P of about 1,000 or smaller.

The log P of many perfume ingredients has been reported; for example, the Pomona 92 database, available from Daylight Chemical Information Systems, Inc. (Daylog CIS), Irvine, Calif., contains many, along with citations to the original literature. However, the log P values are most conveniently calculated by the “C LOG P” program, also available from Daylight CIS. This program also lists experimental log P values when they are available in the Pomona 92 database. The “calculated log p” (C log P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990, incorporated herein by reference). The fragment approach is based on the chemical structure of each perfume ingredient, and takes into account the numbers and types of atoms, the atom connectivity, and chemical bonding. The C log P values, which are the most reliable and widely used estimates for this physicochemical property, are used instead of the experimental log P values in the selection of perfume ingredients which are useful in the present invention.

When hydrophilic perfume is desired, at least about 25% by weight of the perfume, is composed of perfume ingredients having a C log P of about 3 or smaller, in another example at least about 50%, in another example at least about 75%.

In one example, at least about 25%, by weight of the perfume, is composed of fragrance material, in another example at least about 50%, in another example at least about 75%. The fragrance material is selected from the group consisting of aromatic and aliphatic esters having molecular weights from about 130 to about 250; aliphatic and aromatic alcohols having molecular weights from about 90 to about 240; aliphatic ketones having molecular weights from about 150 to about 260; aromatic ketones having molecular weights from about 150 to about 270; aromatic and aliphatic lactones having molecular weights from about 130 to about 290; aliphatic aldehydes having molecular weights from about 140 to about 200; aromatic aldehydes having molecular weights from about 90 to about 230; aliphatic and aromatic ethers having molecular weights from about 150 to about 270; and condensation products of aldehydes and amines having molecular weights from about 180 to about 320; and essentially free from nitromusks and halogenated fragrance materials.

A representative, non-limiting, list of fragrance materials that may be included in the perfume can include anethole, benzaldehyde, decyl aldehyde, benzyl acetate, benzyl alcohol, benzyl formate, benzyl propionate, iso-bornyl acetate, camphene, cis-citral (neral), citronellal, citronellol, citronellal acetate, paracymene, decanal, dihydrolinalool, dihydromyrcenol, methyl benzyl carbinyl acetate, dimethyl benzyl carbinyl acetate, dimethyl phenyl carbinol, eucalyptol, helional, geranial, geraniol, geranyl acetate, geranyl nitrile, cis-3-hexenyl acetate, dihydrocitronellal, d-limonene, linalool, linalool oxide, tetra-hydro linalool, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neral acetate, nonyl acetate, phenyl ethyl alcohol, phenyl acetaldehyde, alpha-pinene, beta-pinene, gamma-terpinene, terpineol, alpha-terpineol, beta-terpineol, terpinyl acetate, vertenex (para-tertiary-butyl cyclohexyl acetate), gamma-methyl ionone, undecalactone, undecylenic aldehyde, alpha-damascone, beta-damascone, amyl acetate, lemon oil, orange oil, and mixtures thereof.

The fragrance material may include the materials delineated above, or may include other perfumes/aromatic materials known to a person of ordinary skill in the art of creating fragrances. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308 and U.S. Pat. No. 4,304,679 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4). Additional fragrance materials can also be found in U.S. Pat. No. 5,939,060 and U.S. patent application Ser. No. 11/712,771.

In one example, the composition comprises from about 0.01% to about 1% perfume, by weight of the composition, in another example from about 0.01% to about 0.5%, and in another example from about 0.015% to about 0.3%. When cyclodextrin is added to the composition, the perfume to cyclodextrin weight ratio is typically from about 3:100 to about 100:100, preferably from about 4:100 to about 50:100, more preferably from about 5:100 to about 40:100, even more preferably from about 5:100 to about 25:100, most preferably from about 1:8 to about 1:4.

### Conditioning Agent

In one example, the deodorizing composition comprises a conditioning agent. Non-limiting examples of conditioning agents can include cottonseed oil, argan oil, linseed oil, grapeseed oil, sunflower oil, olive oil, macadamia nut oil, coconut oil, peppermint oil, lavender oil, rosemary oil, almond oil, tea tree oil, jojoba oil, fatty alcohols, silicones including water soluble silicones, dimethicone, and cyclomethicone, and combinations thereof. Conditioning agents can make hair feel smoother, shinier, and it can reduce static electricity and flyaway hairs.

In one example, the conditioning agent is a water soluble silicone, such as polyalkyleneoxide modified polydimethylsiloxane (commercially available as Silwet®, from Momentive™, Columbus, Ohio). The water soluble silicone can have conditioning benefits for the pet's coat, in particular it can make the coat shiny and soft. The polyalkyleneoxide modified polydimethylsiloxane can be prepared according to the procedure set forth in U.S. Pat. No. 3,299,112. Non-limiting examples of Silwet® surfactants are L-7600, L-7602, L-7604, L-7605, L-7657, and mixtures thereof. In one example, the composition comprises Silwet® L-7600.

The composition can comprise from about 0.0001% to about 0.1% water soluble silicone, in another example from 0.0005% to about 0.05% water soluble silicone, in another example 0.00075% to about 0.01%, and in another example from about 0.0009% to about 0.0025%. In another example, the composition comprises from about 0.25% to about 5% water soluble silicone, in another example from about 0.5% to about 4%, in another example from about 1% to about 3%, and in another example from about 1.5% to about 2.5%.

The water soluble silicone can have an HLB Value, which is the ratio of the hydrophilic to the lipophilic balance and is on a scale of 1-20 with 20 being the most water soluble. The HLB value is calculated as follows:

HLB=20×Mh/M
Where Mh=molecular mass of hydrophobic portion of molecule and
   M=molecular mass of whole molecule

The HLB Value of all of the water soluble silicones can be greater than about 6, in another example greater than about 10, in another example greater than about 12, and in another example greater than about 14. In one example, the HLB Value of all the water soluble silicones is from 5 to 20, in another example 7 to 18, in another example 10 to 17, and in another example 12 to 15.

The molecular weight of the water soluble silicone is from about 500 to about 10,000, in another example from about 1000 to 8000, in another example from about 2000 to about 6000, and in another example from about 3000 to about 5500. In another example, the molecular weight is less than about 7500, in another example less than about 6500, in another example less than about 5500, and in another example less than about 5000.

In one example, the number water soluble silicone comprises ethyleneoxy (EO) units (C₂H₄O) in a polyether chain (R¹). The EO units must be sufficient to render the water soluble silicone water dispersible or water soluble. If propyleneoxy (PO) units are present in apolyalkylenoxy chain, they can be distributed randomly in the chain or exist as blocks. In one example, the water soluble silicone comprises no PO units. In another example, the ratio of EO units to PO units is greater than 1, in another example greater than 10, in another example greater than 100, and in another example greater than 500.

### Solubilizing Aid

In some examples, the deodorizing composition can comprise a solubilizing aid. In particular, a solubilizing agent is selected that can optimize the perfume and therapeutic agent, if present, solubility in the aqueous solution while minimizing the stickiness of the solution when dry. The composition can comprise from about 0.02% to about 3%, by weight of the composition, solubilizing aid, in another example from about 0.05% to about 1%, and in another example from about 0.1% to about 0.3%, by weight of the composition. In another example the composition can comprise from about 0.5% to about 5%, by weight of the composition, solubilizing aid, and in another example from about 0.75% to about 2%, and in another example from about 0.9% to about 1.5%.

In one example, the solubilizing aid can be polyethylene glycol (PEG) functionalized hydrogenated castor oil (commercially available as Basophor® from BASF, Ludwigshafen, Germany). PEG functionalized hydrogenated castor oil can vary based on the number of PEG units that are attached to the hydroxyl group of the castor oil. In one example, PEG functionalized hydrogentated castor oil has from 5 to 100 PEG units, in another example from 10 to 80, in another example from 20 to 50, and in another example from 30 to 40. In one example, the PEG functionalized hydrogenated castor oil has 60 PET units (commercially available as Basophor® ELH60).

In one example, the solubilizing aid can be a surfactant. In some examples, the surfactant, in particular a cationic surfactant, can not only act as a solubilizing aid but it can help protect the hair by binding to the keratin proteins in the hair. However, some surfactants can be irritating so any surfactant that is added to the deodorizing composition must be carefully selected and/or added at a low level.

Suitable surfactants can include nonionic surfactants, cationic surfactants, amphoteric surfactants, zwitterionic surfactants, and mixtures thereof. In one example, the surfactant can be selected from the group consisting of nonionic surfactants, cationic surfactants, and mixtures thereof. In one example, anionic surfactants are not preferred, because they can form water-insoluble salts with the metal ions of the preferred metallic salts of this composition. When a surfactant containing one, or more, alkyl group is used, it is preferred that it contain relatively short alkyl chains of from about 5 to about 14 carbon atoms. Typical nonionic surfactants are polyethylene glycol-polypropylene glycol block copolymers, such as Pluronic® surfactants from BASF, ethoxylated branched aliphatic diols, such as Surfynol® surfactants from Air Products; ethoxylated alkyl phenols, such as Igepal® surfactants from Rhone-Poulene; alcohols and carboxylic acids; polyethylene glycol diesters of fatty acids; and mixtures thereof.

In one example, the solubilizing aid is a nonionic surfactant selected from the group consisting of fatty acid esters of ethoxylated sorbitans. In another example, the solubilizing aid is selected from the group consisting of mixtures of laurate esters of sorbitol and sorbitol anhydrides; mixtures of stearate esters of sorbitol and sorbitol anhydrides; and mixtures of oleate esters of sorbitol and sorbitol anhydrides. In yet another example, the solubilizing aid is selected from the group consisting of Polysorbate 20, which is a mixture of laurate esters of sorbitol and sorbitol anhydrides consisting predominantly of the monoester, condensed with about 20 moles of ethylene oxide; Polysorbate 60 which is a mixture of stearate esters of sorbitol and sorbitol anhydride, consisting predominantly of the monoester, condensed with about 20 moles of ethylene oxide; Polysorbate 80 which is a mixture of oleate esters of sorbitol and sorbitol anhydrides, consisting predominantly of the monoester, condensed with about 20 moles of ethylene oxide; and mixtures thereof.

In one example, the surfactant can have the hydrophilic groups between hydrophobic chains, such as, Pluronic® surfactants, Surfynol® surfactants, polyethylene glycol diesters of fatty acids, fatty acid esters of ethoxylated sorbitans, and mixtures thereof. Inn one example, the surfactant can be polyalkylene glycol.

When a solubilizing aid is used in the composition of the present invention, more specifically when fatty acid esters of ethoxylated sorbitans are used as the solubilizing aid, it is preferable to use the process of high shear milling in order aid in the incorporation of excess hydrophobic organic material.

The solubilizing aids disclosed in the present invention, specifically the fatty acid esters of ethoxylated sorbitan, can be used in any type of composition where excess hydrophobic organic material, particularly perfume, separates out of solution and needs assistance to be incorporated into the composition.

### Buffer System

In one example, the deodorizing composition comprises a buffer system comprising both an acid and a base that is also non-irritating. In one example, the acid can be citric acid and the base can be sodium citrate. Having a buffer system comprising both an acid and a base can provide superior odor neutralization because both acidic and basic malodors can be neutralized.

Non-limiting examples of buffers can include acetate buffers, citrate buffers, phosphate buffers, and combinations thereof. Additional non-limiting examples of buffers can include acetic acid, sodium acetate, citric acid, sodium citrate, monobasic sodium phosphate, dibasic sodium phosphate, sodium carbonate, sodium bicarbonate, succinic acid, sodium succinate, potassium dihydrogen phosphate, phosphoric acid, and combinations thereof.

In one example, the deodorizing composition is substantially free of triethanolamine. In another example the composition comprises less than about 100 ppm, in another example less than about 50 ppm, in another example less than 25 ppm, and in another example less than about 15 ppm.

Bacteriostatic effects can sometimes be obtained for aqueous compositions by adjusting the composition pH to an acid pH, e.g., less than about pH 4 or less than about pH 3. In one example, low pH for microbial control is not a preferred approach in the present invention because the low pH can cause chemical degradation of the cyclodextrins, as well as irritate the pets' skin. Therefore, aqueous compositions of the present invention should have a pH greater than about 4.0, in another example greater than about 5.0, and in another example greater than about 6.0. In one example the pH is from about 4.0 to about 9.0, in another example from about 5.0 to about 8.5, and in another example from about 6.0 to about 8.0. In one example, the composition has a pH of about 7.0.

### Metallic Salt

In one example, the deodorizing composition can comprise a metallic salt, for example water-soluble zinc salts. A water-soluble metallic salt can be present in the deodorizing composition of the present invention to absorb amine and sulfur-containing compounds thereby helping to further reduce malodor. The water-soluble metallic salts can be selected from the group consisting of copper salts, zinc salts, and mixtures thereof.

Non-limiting examples of metallic salts can include water-soluble zinc salts are zinc chloride, zinc gluconate, zinc lactate, zinc maleate, zinc salicylate, zinc sulfate, copper chloride, copper gluconate, and combinations thereof.

In one example, the composition comprises from about 0.1% to about 10% metallic salts, by weight of the composition, in another example from about 0.2% to about 7%, in another example from about 0.3% to about 5%.

### Aqueous Carrier

The deodorizing composition can be an aqueous solution. In one example, the aqueous carrier can be water. The water can be distilled, deionized, or tap water. In another example, the aqueous carrier can be a low molecular weight monohydric alcohol, e.g., ethanol; methanol, and isopropanol, or polyols, such as ethylene glycol and propylene glycol.

In another example, the aqueous carrier can comprise both water and a low molecular weight monohydric alcohol, such as ethanol. The ethanol can help the deodorizing composition dry faster after it has been applied. However, pets, especially small pets, can be particularly sensitive to alcohol and if too much is added to the solution, even a couple of licks through self grooming can potentially harm the pet.

In one example, the composition can comprise about 80% to about 99%, by weight, aqueous carrier, in another example about 90% to about 97%, in another example about 93% to about 96%. In another example, the composition can comprise from about 75% to about 97%, by weight, water, in another example 85% to about 95%, and in another example from about 88% to about 92%. In another example, the composition can comprise from about 1% to about 10%, by weight, monohydric alcohol, in another example from about 2% to about 7%, and in another example from about 3% to about 6%.

### Preservative

The deodorizing composition can optionally comprise water-soluble preservatives. A preservative can be preferred when the deodorizing composition is stored in a container that can be opened or exposed to the air. This is because cyclodextrin molecules are made up of varying numbers of glucose units, which can make them a prime breeding ground for certain microorganisms, especially when in an aqueous composition. Because microbial growth in cyclodextrin solutions is highly objectionable when it occurs, it can be preferable to include a solubilized water-soluble, antimicrobial preservative, which is effective for inhibiting and/or regulating microbial growth in order to increase storage stability of the preferably clear, aqueous odor-absorbing solution containing water-soluble cyclodextrin.

However, some preservatives can irritate a pet's sensitive skin and/or pose risks when ingested. In one example, especially in an example where the deodorizing composition is stored in a closed container, the deodorizing composition is substantially free of a preservative.

If present, the concentration and type of preservative must be carefully selected because some preservatives can be irritating to a pet's skin. Furthermore, the preservative should not significantly interfere with the malodor neutralizing and entrapping properties of the composition. The preservative can be water-soluble. In one example, the preservative level is less than about 0.01%, by weight of the composition, in another example less than 0.005%, and in another example less than 0.0025%. In one example the composition comprises from about 0.0001% to about 0.1%, by weight, preservative, in another example from about 0.0005% to about 0.02%, and in another example from about 0.00075% to about 0.0025%.

In one example, the composition is substantially free of phenoxyethanol. In one example, the composition comprises less than about 50 ppm phenoxyethanol, in another example less than about 25 ppm, in another example less than about 10 ppm, in another example less than about 5 ppm, and in another example less than about 1 ppm.

In one example, a broad spectrum preservative, e.g., one that is effective on both bacteria (both gram positive and gram negative) and fungi can be used

The antimicrobial preservatives can be biocidal compounds, i.e., substances that kill microorganisms, or biostatic compounds, i.e., substances that inhibit and/or regulate the growth of microorganisms.

Non-limiting examples of preservatives can include 2-methyl-4-isothiazolin-3-one (MIT) (commercially available as Acticide™ M20, Thor GmbH in Speyer, Germany), 1,2-benzisothiazolin-3-one (BIT) (commercially available as Proxel™ GXL, Arch Chemicals, Basel Switzerland), benzalkonium chloride, ethylenediaminetetraacetic acid (EDTA), benzyl alcohol, potassium sorbate, parabens, benzoic acid, sodium benzoate, phenoxyethanol, ethylhexylglycerine, natural preservatives, and mixtures thereof. Non-limiting examples of natural preservatives can include rosemary extract, hops, Vitamin E, Vitamin C, ascorbic acid from lemon or other citrus juice, and combinations thereof. In one example, the preservative is MIT.

Additional non-limiting examples of preservatives can include organic sulfur compounds, cyclic organic nitrogen compounds, low molecular weight aldehydes, quaternary compounds, dehydroacetic acid, phenyl and phenoxy compounds, and mixtures thereof.

In one example, the preservative can be water-soluble. Water-soluble preservatives can have a solubility in water of at least about 0.3 g per 100 ml of water, i.e., about 0.3% at room temperature, and in another example greater than about 0.5% at room temperature.

Non-limiting examples of the water-soluble preservatives can include a mixture of about 77% 5-chloro-2-methyl-4-isothia-zolin-3-one and about 23% 2-methyl-4-isothiazolin-3-one, a broad spectrum preservative available as a 1.5% aqueous solution (commercially available as Kathon™, Rohm and Haas, Philadelphia, Pa.), 5-bromo-5-nitro-1,3-dioxane (commercially available as Bronidox® L, Cognis, Monheim, Germany), a 95:5 mixture of 1,3-bis(hydroxymethyl)-5,5-dimethyl-2,4-imidazolidinedione and 3-butyl-2-iodopropynyl carbamate (commercially available as Glydant® Plus Liquid, Lonza, Basel, Switzerland), diazolidinyl urea imidazolidinyl urea, glutaraldehyde, polyaminopropyl biguanide (PAPB), and mixtures thereof.

### Therapeutic Agents

In one example, the deodorizing composition can comprise one or more therapeutic agents.

In one example the therapeutic agent is a parasiticide treatment to prevent fleas, ear mites, hookworms, roundworms, heartworms, sarcoptic mange, and lice. In another example, the therapeutic agent helps reduce irritation of the pet's skin and can include a moisturizer, and/or an anti-itch medication.

In another example, the deodorizing composition can comprise an antibiotic, anti-fungal, and/or anti-yeast composition, which can treat the naturally occurring, odor causing yeast and bacteria or can be used to treat or prevent an infection.

In another example, the deodorizing composition can comprise sunscreen to protect the hair and/or skin from sun damage.

In another example, the deodorizing composition can comprise reconstructors, such as hydrolyzed protein, to aid in strengthening the hair structure.

In another example, the deodorizing composition can comprise a therapeutic agent that reduces hair loss and/or promote hair regrowth such as minoxidil.

In another example, the deodorizing composition can comprise thermal protectors, such as heat absorbing polymers, to help shield the hair against excessive heat such as heat from a blow dryer or curling iron.

In another example, the therapeutic agent can be an oral care agent such as zinc, copper, and/or sodium hexametaphosphate, and salts thereof. These therapeutic agents can enter the oral cavity through self-grooming.

In another example, the deodorizing composition can comprise a soil capturing polymer such as Mirapol® (commercially available from Rhodia, Bristol, Pa.) or other soil capture polymers. The soil capturing polymer can be in the deodorizing composition and/or they can be deposited on the dirt lifter. These polymers are highly charged and can attract odor causing agents, especially dirt, dust, and hair.

### Dispenser

The deodorizing composition can be dispensed by any suitable means. Non-limiting examples of dispensers can include pre-moistened cloths, a bottle, a squeeze bottle, a spray bottle, or combinations thereof.

In one example, the dispenser can be a spray bottle. The composition is placed into a spray dispenser in order to be distributed onto the pet or onto a dirt lifter and then dispersed across the pet's coat. The spray dispenser is any of the manually activated means for producing a spray of liquid droplets as is known in the art, e.g. trigger-type, pump-type, non-aerosol self-pressurized, and aerosol-type spray means.

The spray dispenser can be an aerosol dispenser. A continuous action aerosol spray can be easier for a consumer to use, especially when a consumer frequently has at least one hand on their pet while spraying. Said aerosol dispenser comprises a container which can be constructed of any of the conventional materials employed in fabricating aerosol containers including but not limited to aluminum, stainless steel, and other metals and plastic. The dispenser must be capable of withstanding internal pressure in the range of from about 20 to about 110 p.s.i.g., more preferably from about 20 to about 70 p.s.i.g. The dispenser can have a valve member that will permit the deodorizing composition contained in the dispenser to be dispensed in the form of a spray of very fine, or finely divided, particles or droplets. The aerosol dispenser utilizes a pressurized sealed container from which the deodorizing composition is dispensed through a special actuator/valve assembly under pressure. The aerosol dispenser is pressurized by incorporating therein a gaseous component generally known as a propellant. Non-limiting examples of propellants can include compressed air, nitrogen, inert gases, carbon dioxide, and combinations thereof. In one example, the propellant is nitrogen. A more complete description of commercially available aerosol-spray dispensers appears in U.S. Pat. Nos. 3,436,772 and 3,600,325.

In another example, the spray dispenser can be a self-pressurized non-aerosol container having a convoluted liner and an elastomeric sleeve. Said self-pressurized dispenser comprises a liner/sleeve assembly containing a thin, flexible radially expandable convoluted plastic liner of from about 0.010 to about 0.020 inch thick, inside an essentially cylindrical elastomeric sleeve. A more complete description of self-pressurized spray dispensers can be found in U.S. Pat. Nos. 5,111,971 and 5,232,126.

Another type of aerosol spray dispenser is one wherein a barrier separates the odor absorbing composition from the propellant (preferably compressed air or nitrogen), as disclosed in U.S. Pat. No. 4,260,110.

In another example, the spray dispenser can be a non-aerosol, manually activated, pump-spray dispenser. Said pump-spray dispenser can comprise a container and a pump mechanism which securely screws or snaps onto the container. The container can comprise a vessel for containing the aqueous deodorizing composition to be dispensed.

The pump mechanism comprises a pump chamber of substantially fixed volume, having an opening at the inner end thereof. Within the pump chamber is located a pump stem having a piston on the end thereof disposed for reciprocal motion in the pump chamber. The pump stem has a passageway there through with a dispensing outlet at the outer end of the passageway and an axial inlet port located inwardly thereof.

In another example, the spray dispenser is a manually activated trigger-spray dispenser. Said trigger-spray dispenser can comprise a container and a trigger. The trigger-spray dispenser herein can be one which acts upon a discrete amount of the deodorizing composition itself, typically by means of a piston or a collapsing bellows that displaces the composition through a nozzle to create a spray of thin liquid. Said trigger-spray dispenser typically comprises a pump chamber having either a piston or bellows which is movable through a limited stroke response to the trigger for varying the volume of said pump chamber. This pump chamber or bellows chamber collects and holds the product for dispensing.

The container, including the container for an aerosol spray bottle, and the pump mechanism, if present, can be constructed of any conventional material employed in fabricating spray bottles, including, but not limited to: polyethylene, polypropylene, polyacetal, polycarbonate, polyvinyl chloride, polyethyleneterephthalate, blends of polyethylene, polystyrene, vinyl acetate, rubber elastomer, stainless steel, glass, and combinations thereof. In one example, the container is made of clear, e.g., polyethylene terephthalate. In one example, the bottle is made out of stainless steel. Additional information on commercially available dispensing devices appears in U.S. Pat. Nos. 4,895,279, 4,735,347, and 4,274,560.

Additional information on spray bottles and dispensing devices can be found in U.S. Pat. Nos. 4,082,223, 4,161,288, 4,434,917, and 4,819,835, and 5,303,867.

### Dirt Lifter

In some examples, the composition can be sprayed directly on the pet and allowed to dry or wiped with a towel or paper towel. However, in some examples, to help remove odor causing agents, a dirt lifter can be used in combination with the deodorizing composition. The deodorizing composition can be dispensed directly on the pet or it can be dispensed on the dirt lifter and then distributed across the pet with the dirt lifter. Dispensing the deodorizing composition on the dirt lifter and then distributing it across the pet's coat can be preferable since some pets dislike being sprayed with a liquid.

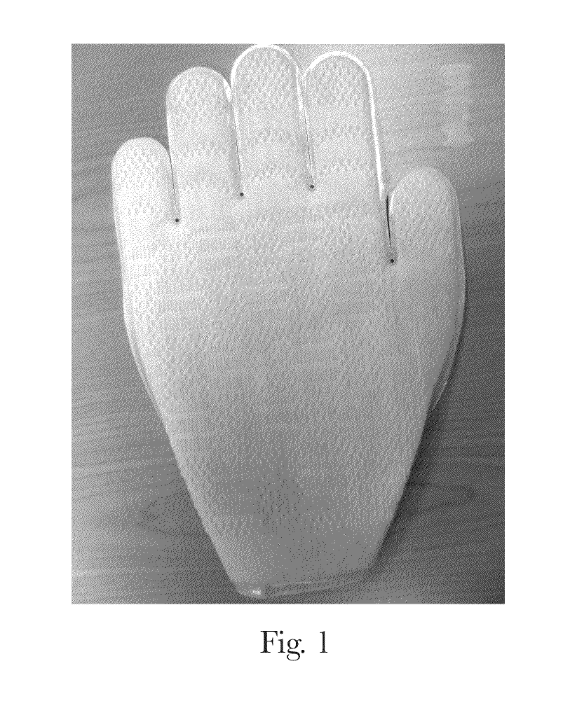

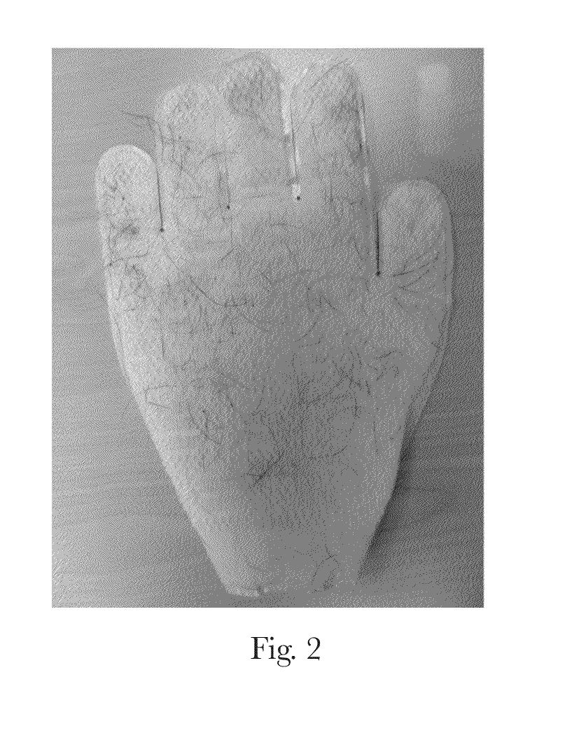

As seen in FIGS. 1 and 2, the dirt lifter can remove odor causing agents from the pet. FIG. 1 shows a dirt lifter, in the form of a glove, before it was used in combination with the deodorizing composition. FIG. 2 shows the same dirt lifter after it was used to deodorize and clean a dog. FIG. 2 clearly shows that the dirt lifter collected dirt and hair during use. In this example, some of the dirt collected on the tops of the tufts. The fingers are also especially dirty because the user used her fingers to massage deep into the dog's coat and remove odor causing agents.

The dirt lifter can also provide a visual indicator that the pet is cleaner and/or deodorized. In one example, the dirt lifter is a light color, such as white or cream, and the user can see the dirt and hair that have been removed by the dirt lifter. In another example, the dirt lifter can be a first color in a first region, such as the finger region, and a second color in a second region, such as the palm region.

In one example, only one dirt lifter is used to clean the pet. In another example, two dirt lifters are used simultaneously, with one on each hand. Two dirt lifters can be preferred for large pets. Surprisingly, it has been found that users can more easily penetrate the coat and disperse the deodorizing composition on a large pet if they are using two dirt lifters.

The dirt lifter can be any suitable shape or made out of any suitable material that can distribute the deodorizing composition and/or attract dirt. In one example, the dirt lifter can be a sheet, preferably a laminate sheet.

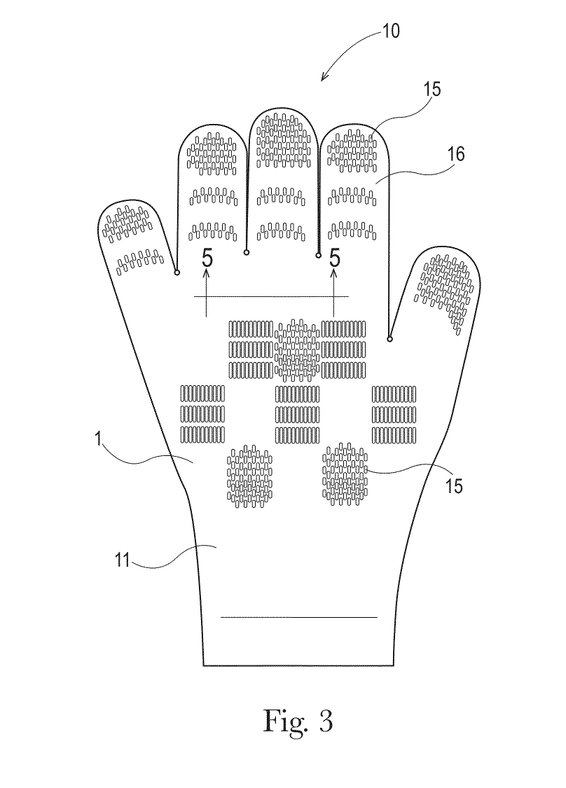

In another example, the dirt lifter can be an on-the-hand implement, such as a dirt lifting glove 10. FIG. 3 shows front side 1 of dirt lifting glove 10.

Facing sheet 11 can include textured portions 15. The textured portions can provide superior cleaning by providing additional surface area and abrasion and can more effectively penetrate the pet's coat than a surface that is flat or relatively flat. Surprisingly, it has been found that a textured dirt lifter can remove more odor causing agents than a dirt lifter that is essentially flat. The texture can be any texture including protrusions, depressions, apertures, embossing, debossing, and combinations thereof. In one example, the textured portion can comprise tufts that can be made by SELFing. As used herein, “SELF” or “SELFing”, refer to Procter & Gamble technology in which SELF stands for Structural Elastic Like Film. Processes, apparatus, and patterns produced via SELF are illustrated and described in U.S. Pat. Nos. 5,518,801; 5,691,035; 5,723,087; 5,891,544; 5,916,663; 6,027,483; and, 7,527,615.

The textured portions can be on the entire dirt lifter or only a portion of the dirt lifter. The textured portions can have any pattern. In one example, the textured portions can be rigid and will not deform during normal use on a pet. In another example, the dirt lifter can be made out of resilient material that does not rip, shred, or otherwise break during normal use.

The textured portions can be the same or vary across facing sheet 11. For instance, textured portions 15 on the fingers and thumb can be designed to help remove odor causing agents, especially agents that are trapped in the coat. In one example, the areas designated for the fingers and thumb of the glove can comprise knuckle regions 16, which are areas that have a different texture or a smooth texture that can indicate that the fingers and thumb are flexible. When the user understands that the fingers and thumbs are flexible she will use her fingers and thumb to massage the pet and can more easily remove odor causing agents that is lodged in the coat. Textured portions 15 that are located on the palm can be designed to help spread the deodorizing composition. The textured portions on the palm can be longer than the textured portions on the fingers and thumb.

The number, spacing, and dimensions of textured portions 15 can be the same or they can be varied to give varying texture to front side 1. For example, if textured portions 15 are closely spaced, front side 1 can have a terry cloth-like feel. Alternatively, textured portions 15 can be arranged in patterns such as lines or filled shapes to create portions having greater texture, softness, bulk, absorbency or visual design appeal. For example, when textured portions 15 are arranged in a pattern of one or more lines, textured portions 15 can have the appearance of stitching. Textured portions 15 can also be arranged to form specific shapes, such as designs, words or logos. In one example, the textured portions 15, especially those located on the palm, can provide a visual indication that the dirt lifter is made out of an absorbent material. In one example, textured portions 15, can cover the entire palm and can be in a pattern that looks quilted.

In one example, the textured portions can be protrusions, including tufts, and the size dimensions, such as the height, length and width of the individual protrusions can be varied. In one example, the protrusions have a height from about 0.5 mm to 5 mm, in another example from about 0.75 mm to about 3 mm, in another example from about 1 mm to about 2.5 mm, and in another example from about 1.5 mm to about 2.25 mm. In another example, the protrusions have a length of from about 1 mm to about 6 mm, in another example from about 2 mm to about 4 mm, and in another example from about 2.5 mm to about 3.5 mm. In another example, the protrusions can have a length of from about 4 mm to about 10 mm, in another example from about 5 mm to about 8 mm, and in another example from about 6 mm to about 7 mm. In another example, the distance between protrusions or tufts can be from about 1 mm to about 10 mm, in another example from about 2 mm to about 7 mm, and in another example from about 3 mm to about 5 mm. In another example, the height of the length and height of the textured portions can be customized based on the length of the pet's coat.

The number of protrusions per unit area of front side 1, i.e., the area density of the protrusions, can be the same or varied. The area density can be from about 1 protrusion per unit area, e.g., square centimeter to as high as about 100 protrusions per unit area. In one example, there can be at least about 10 protrusions, or at least about 20 protrusions per square centimeter. The area density does not need to be uniform across front side 1.

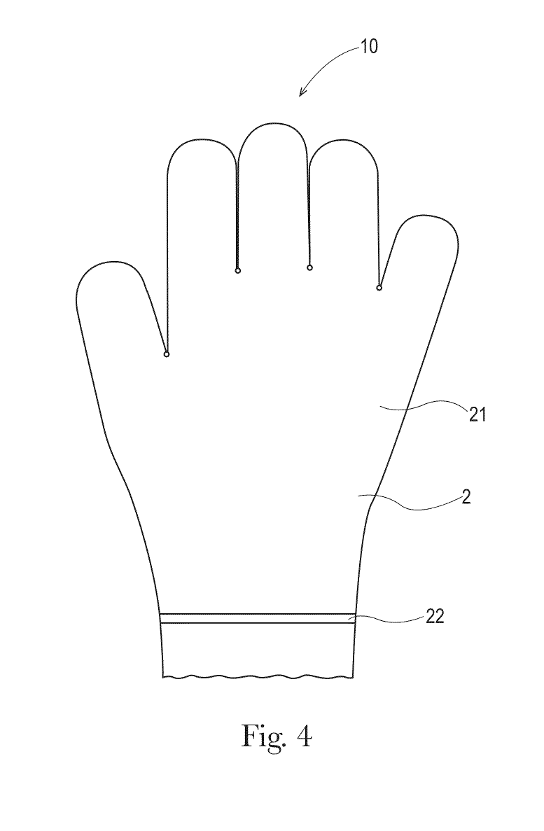

FIG. 4 shows back side 2 of dirt lifting glove 10. Back side 2 can include backing sheet 21 and adjustment means 22. Back side 2 can also have adjustment means 22, which can help dirt lifting glove 10 accommodate a variety of hand sizes. When the on-the-hand implement fits correctly, it can make using the dirt lifter easier and can allow for controlled movements, similar to petting or massaging a pet. Non-limiting examples of suitable adjustment means, can include hook and loop fasteners such as Velcro® and the like, elastic members, elastic strands, strings including draw strings, gussets, cinches, buttons, fasteners, tabs, tongue and grove fasteners such as Zip-Lock® type fasteners, resealable tape, belts, clips, adhesives, such as refastenable adhesives, and combinations thereof. In one example, adjustment means 22 can be made out of elastane or other elastic.

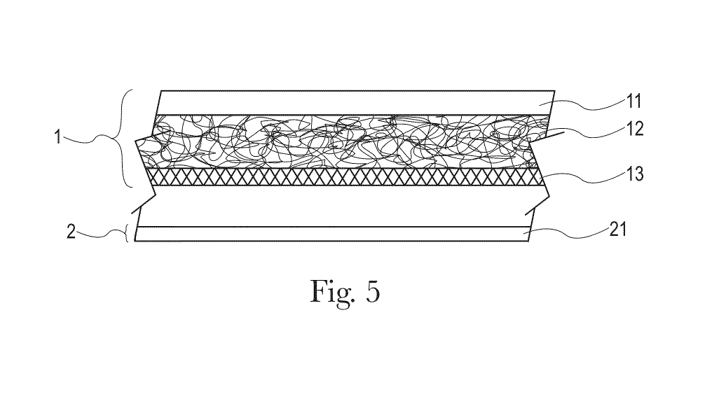

As shown in FIG. 5, front side 1 can comprise facing sheet 11, absorbent sheet 12, and inner sheet 13 and back side 2 can comprise backing sheet 21.

Facing sheet 11 can be made out of any suitable material. In one example, facing sheet 11 can be made out of a nonwoven. The nonwoven can be permeable so the deodorizing composition penetrates facing sheet 11 and is absorbed into absorbent layer 12. In another example, the nonwoven can be made out of a wicking material that will direct the deodorizing composition into the nonwoven. The material of the facing sheet can be apertured or otherwise have gaps that allow odor causing agents to become lodged on the surface and can allow the deodorizing composition to pass through. As used herein, the term “nonwoven web” or “nonwoven” refers to a web having a structure of individual fibers or threads which are interlaid, but not in a repeating pattern as in a woven or knitted fabric, which do not have randomly oriented fibers. Nonwoven web or fabrics can be formed from many known processes.

Absorbent sheet 12 can be made out of any suitable material. In one example, absorbent sheet 12 can be made out of a material that can allow for the liquid deodorizing composition to be held but does not capture the liquid composition indefinitely and can release it when the dirt lifter is in use. Some materials, can be too absorbent and thus need to be supersaturated before they can be used to disperse a liquid, these materials can be used but are less preferred. However, if the material isn't absorbent enough, then it will drip and the user will not be able to control the application of the liquid to the pet. In one example, the absorbent sheet can be made out of a material with a random array of void spaces throughout its structure, such as high loft batting material. In addition to helping to hold and release the liquid composition, the void spaces can compress, allowing the dirt lifter to be conformable and/or flexible. In one example, the absorbent layer does not comprise cellulosic fibers. In another example, the absorbent layer comprises cellulosic fibers in combination with polyethylene terephthalate fibers and/or polypropylene fibers.

In one example, the high loft nonwoven can have a density of about 20 g/m² to about 150 g/m², in another example from about 40 g/m² to about 110 g/m², in another example from about 60 g/m² to about 100 g/m², and in another example from about 70 g/m² to about 90 g/m².

Inner sheet 13 can be made out of any suitable material. In one example, inner sheet 13 can be impermeable to the deodorizing composition and an impermeable sheet can help to spread the deodorizing composition because the composition is not absorbed onto the user's hand. In another example, inner sheet 13 is semi-impermeable.

Back side 2 can include backing sheet 3. In one example, backing sheet 3 can be made out of a material that is extensible. In another example, backing sheet 3 can comprise stretchy fibers such as elastane fibers. In another example, backing sheet 3 can be made out of a nonwoven that comprises a SELF web that readily fits and conforms to a wearer's hand, as described in WO 97/38598.

Front side 1 and back side 2 are joined together at the perimeter. In one example, front side 1 and back side 2 are joined around the entire perimeter, except for the portion adapted for the hand to enter the dirt lifter. Front side 1 and back side 2 can be joined by any suitable method. In one example, the two sides are joined by heat sealing. Alternatively, the two sides can be joined by mechanical sealing and/or with adhesive.

In one example, the dirt lifter can attract odor causing agents. In one example, the dirt lifter attracts odor causing agents because it is wet while in use. The dirt lifter can be wet because it is sprayed with the deodorizing composition or absorbs the deodorizing composition during use. In another example, the dirt lifter can be sold as a wet product and can be pre-moistented with the deodorizing composition, such as a pre-moistened wipe.

In another example, the dirt lifter can be used without a liquid deodorizing composition. The dirt lifter can be made from an electrostatic fabric that when you rub the dirt lifter across the pet, it develops a static charge and the odor causing agents, in particular hair and small dirt particles, develop a static charge and are attracted to the dirt lifter. The dirt lifter can also be coated with a composition, such as a microcrystalline wax, that is sticky and/or attracts odor causing agents.

The dirt lifter can comprise indicia. The indicia can be in the form of logos, trademarks, geometric patterns, images of the surfaces that the implement is intended to clean, instructions on how to use the dirt lifter including the function or purpose of one or both sides of the implement, other indicia, or combinations thereof. In another example, the dirt lifter can comprise a wetness indicator. The wetness indicator can be indicia and/or coloration that can show when to apply more deodorizing composition and/or when enough deodorizing composition has been applied to the dirt lifter.

The dirt lifter can be disposable. As used herein, “disposable” refers to an implement that is disposed or discarded after a limited number of usage events. In one example, the disposable dirt lifter is used five or fewer times, in another example three or fewer times, in another example two or fewer times, and in another example the dirt lifter is used only one time. In one example, a user will deodorize multiple pets before disposing of the dirt lifter and in another example the user will only deodorize one pet before disposing of the dirt lifer.

The dirt lifter can be any suitable material and the material and can be selected based on the pet's coat. The softness or stiffness of the sheets can be changed depending on the basis weight of the constituent material, the size of the denier fibers, and the strength or stiffness of the constituent material.

In certain examples, the sheets can be nonwovens (i.e. a natural or synthetic nonwovens including fibrous and nonfibrous nonwovens), wovens, films (e.g. a formed film), sponges (e.g. a natural or synthetic sponge), polymeric netted meshes (i.e. a “scrim”), batting, spunbound, spunlace, hydroentangled, carded, needlepunch, or any other suitable material. In one example, the sheets can be fibrous nonwovens.

In one example, one or more sheets can comprise nonwovens that can be made out of a plurality of fibers which can include natural fibers, synthetic fibers, and combinations thereof. In another example, the nonwovens can be an airlaid nonwoven material comprising a combination of natural fibers, staple length synthetic fibers and a latex binder. The nonwoven material can be about 20% to about 80% by weight wood pulp fibers, about 10% to about 60% by weight staple length polyester fibers, and about 10% to about 25% by weight binder.

In another example, any of the sheets can comprise an elastomer. The elastomer can be elastic strands and/or an elastic film. Any suitable elastic film known in the art can be used. Suitable elastic films may comprise polypropylene, polyethylene, polyolefins, styrene-isoprene-styrene, styrene-butadiene-styrene, or combinations thereof. Suitable elastic strands can be made of a resilient elastic thermoplastic material. Additional information on elastomers can be found in U.S. Pat. No. 7,432,413 and U.S. patent application Ser. No. 12/687,527.

In another example, the sheets can be biodegradable. For example, the implement or components thereof could be made from a biodegradable material, such as a polyesteramide.

Suitable natural fibers for constructing the nonwoven webs can include but are not limited to cellulosic fibers, such as wood pulp fibers, cotton, and rayon. Suitable synthetic fibers include fibers commonly used in textiles, including but not limited to polyester (e.g. polyethylene terephthalate) and polypropylene, polyethylene, polyether, and combinations thereof. Suitable fibers can be made of biopolymers made from non-petroleum sources such as bio-derived polyethylene (bio-PE), bio-derived polypropylene (bio-PP), bio-derived polyethylene terephthalate (bio-PET), and bio-derived poly(ethylene-2,5-furandicarboxylate) (bio-PEF). These materials can be partially or completely derived from at least one renewable resource where a renewable resource refers to a natural resource that can be replenished within a 100 year time frame. Renewable resources include plants, animals, fish, bacteria, fungi, and forestry products and may be naturally occurring, hybrids, or genetically engineered organisms. Natural resources such as crude oil, coal, and peat which take longer than 100 years to form are not considered to be renewable resources. Other polymers derived from non-petroleum sources include starch-based polymers and cellulosics. Additionally, recycled resins such as post-consumer regrind r-HDPE, r-LLDPE, r-LDPE, r-PET, r-PEF, or r-PP can be used at 100% or blended with various resins. Polymers derived from renewable resources and recycled resins could be used on their own, or blended into petroleum-based polymers at varying levels in order to control the cost. Sources and methods of making polymers from non-petroleum sources can be found in U.S. App. No. 2011/0319849.

Various methods can be used to form the sheets for use in the present invention. For instance, if the sheets are nonwoven webs they can be made by nonwoven dry forming techniques, such as air-laying, or alternatively by wet laying, such as on a papermaking machine, of a continuous web out of which the nonwoven webs are made. Other nonwoven manufacturing techniques, including but not limited to techniques such as spunbonding, meltblowing, carding, needle punching, hydroentangling, thermal bonding, through-air bonding, lamination methods may also be used, and combinations thereof.

The sheets can be subjected to various treatments, such as, but not limited to, physical treatment, such aperturing, embossing, ring rolling SELFing and the like; chemical treatment, such as, rendering part or all of the sheet hydrophobic, and/or hydrophilic, and the like; thermal treatment, such as softening of fibers by heating, thermal bonding and the like; and combinations thereof.

As used herein, the term “aperture”, refers to a hole. The apertures can either be punched cleanly through the sheets of the dirt lifter, described hereafter. The material surrounding the aperture lies in the same plane as the sheet prior to the formation of the aperture (a “two dimensional” aperture), or holes formed in which at least some of the material surrounding the opening is pushed out of the plane of the sheet. In the latter case, the apertures may resemble a protrusion or depression with an aperture therein, and may be referred to herein as a “three dimensional” aperture, a subset of apertures. The apertures can be created by any suitable means including die cut holes, slitting and stretching the sheets and/or substrate, and combinations thereof. The dirt lifter can comprise one or more sheets comprising apertures.

Additional information on materials which are suitable for use as the sheets of the present invention can be found in the following patents: U.S. Pat. No. 3,862,472; U.S. Pat. No. 3,982,302; U.S. Pat. No. 4,004,323; U.S. Pat. No. 4,057,669; U.S. Pat. No. 4,097,965; U.S. Pat. Nos. 4,176,427; 4,130,915; U.S. Pat. No. 4,135,024; U.S. Pat. No. 4,207,367; U.S. Pat. No. 4,296,161; U.S. Pat. No. 4,682,942; U.S. Pat. No. 4,637,859; U.S. Pat. No. 5,223,096; U.S. Pat. Nos. 5,240,562; 5,556,509; and U.S. Pat. No. 5,580,423.

Additional information regarding nonwoven laminate cleaning implements can be found in the following patent applications: U.S. Pat. App. Nos. 61/836,205 and 61/836,209.

### Method of Use

In order to use the deodorizing composition the consumer can distribute the composition throughout the portion of the pet that she wants to clean or across the entire pet. The deodorizing composition can be contained in a spray bottle and an effective amount of deodorizing composition can be dispensed directly on a pet or it can be dispensed on a dirt lifter and then distributed across the pet's coat.

Optionally, a consumer can use a dirt lifter to distribute the cleaning composition across the pet's coat or the portion of the pet's coat she wants to clean and/or deodorize. In another example, the consumer sprays the deodorizing composition directly onto the pet's coat and then uses a dirt lifter to further distribute the composition and to help remove dirt, fur, and other causes of malodor.

This process can be repeated until the pet entire pet is cleaned and deodorized. In order to clean and/or deodorize the user will repeat the process from about 1 to about 7 times, in another example from about 2 to about 5 times, and in another example from about 2 to about 4 times.

In order to deodorize a small pet a user will use from about 1 g to about 7 g of deodorizing composition, in another example from about 2 g to about 5 g, and in another example from about 3 g to about 4.5 g. In order to deodorize a medium pet a user will use from about 3 g to about 11 g of deodorizing composition, in another example from about 4 g to about 8 g, and in another example from about 5 g to about 6 g. In order to deodorize a large pet a user will use from about 4 g to about 15 g of deodorizing composition, in another example from about 5 g to about 10 g, and in another example from about 6 g to about 8 g. In one example, the user will use at least about 2 g to deodorize her pet, in another example, at least about 3 g, and in another example at least about 4 g.

The deodorizing composition can be applied as needed and is often applied in between baths. In one example, the deodorizing composition is applied daily, in another example 2-3 times per week, in another example 3-4 times per week, in another example weekly, and in another example bi-weekly. In another example, the deodorizing composition can be applied during or immediately after a bath.

In one example the deodorizing composition and the dirt lifter can be co-packaged and in another example the deodorizing composition and the dirt lifter can be sold separately. In one example, the deodorizing composition and the dirt lifter can be co-packaged in a starter kit that is intended for consumers who have not previously used the product and also sold separately.

### Example 1

### Deodorizing Composition Intended for Use on Pets

| Components: | Wt. % |
|---|---|
| Deionized Water | 90.587 |
| Non-denatured Ethanol | 5.000 |
| Polyalkyleneoxide | 0.001 |
| modified | |
| polydimethylsiloxane | |
| Sodium Citrate | 0.659 |
| MIT | 0.010 |
| Citric Acid | 0.043 |
| Perfume | 0.450 |
| PEG functionalized | 1.000 |
| hydrogenated castor oil | |
| Hydroxypropyl Beta | 2.250 |
| Cyclodextrin | |

Example 1 was made according to the following method. First, a perfume premix was made by combining PEG functionalized hydrogenated castor oil with the perfume. Once combined, the perfume premix was set aside. First, a mixing vessel was charged with an appropriate amount of deionized water. Then stirring began. The ethanol was added to mixing vessel and stirring continued. Once the ethanol was combined, polyalkyleneoxide modified polydimethylsiloxane, was added, the pH was checked, and stirring continued. Then sodium citrate was added, the pH was checked and stirring continued for five minutes. Then, MIT was added and stirring continued. Then citric acid was added, the pH was checked, and stirring continued for five minutes. Then the perfume premix was added, the pH was checked, and stirring continued for five minutes. Finally the cyclodextrin was added and the mixture was stirred for 15 minutes. The final pH was checked and was about 7.0.

### Example 2

### Deodorizing Composition Intended for Use on Humans

Ninety-nine grams of deionized water were added to 1.0 grams of konjac gum (commercially available from TicGums, Inc., White Marsh, Md.). Agitation was applied to the water in order to form a vortex and the powder was added at a rate that was able to wet the individual particle without the formation of aggregates.

To 99 grams of deionized water was added 1.0 grams of xanthan gum (commercially available as Novaxan Xanthan Gum 174960 from Archer Daniels Midland Co., Decatur, Ill.). Agitation was applied to the water in order to form a vortex and the powder was added at a rate that was able to wet the individual particle without the formation of aggregates.

Next, 30 grams of the 1 wt % solution of konjac gum (preparation as described above) was added to 344 grams deionized water, followed by 20 grams of the 1 wt % solution of xanthan gum (preparation as described above). The contents were mixed to yield a homogeneous, transparent liquid that demonstrated a yield stress. Then, six grams of perfume microcapsule, (15 wt %-60 wt % perfume activity), were added to the formulation while mixing. Finally, 0.12 grams of Kathon CG preservative were added.

This sprayable formulation was then applied to hair switches. Approximately 0.40 grams of the spray was applied to a 4 gram/8 inch (20.32 cm) long hair switch. The hair was allowed to dry for four hours at ambient conditions. Expert perfumers graded the hair switch before and after combing. The same hair switch was left to stand for 24 hours. Expert perfumers graded the hair switch again, before and after combing. The perfumers provided an olfactive intensity grade, as seen below (range 0-100, where a 10 point delta generated by combing is a noticeable different in fragrance intensity).

| Encapsulated | | 4 hr dry hair | 24 hr dry hair |
|---|---|---|---|
| Perfume | | (Pre/Post- | (Pre/Post- |
| (% perfume) | Treatment | Comb) | Comb) |
| 0.20% | 0.40 g on dry hair | 40/60 | 35/55 |
| 0.20% | 0.40 g on damp hair | 45/70 | 40/65 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorizing composition that entraps and neutralizes malodors on a hair covered surface of a mammal comprising:
a. a cyclodextrin;
b. an aqueous carrier;
c. a water soluble silicone with an HLB value that is greater than about 6; and
d. a buffer system comprising citric acid;
wherein the deodorizing composition has a pH from about 5 to about 8.5 and
wherein the deodorizing composition is non-irritating and non-toxic to mammals.

2. The deodorizing composition of claim 1 wherein the buffer system further comprises sodium citrate.

3. The deodorizing composition of claim 1 wherein the pH is from about 6 to about 8.

4. The deodorizing composition of claim 1 wherein the aqueous carrier comprises water and from about 2% to about 7% of a monohydric alcohol.

5. The deodorizing composition of claim 1 comprising from about 0.0005% to about 0.05% water soluble silicone.

6. The deodorizing composition of claim 1 wherein the composition is substantially free of a preservative.

7. The deodorizing composition of claim 1 further comprising a perfume and wherein the composition has a noticeable fresh impression for greater than six hours.

8. The deodorizing composition of claim 1 wherein the mammal is a pet.

9. A deodorizing composition that entraps and neutralizes malodors on a pet comprising:
a. a cyclodextrin;
b. an aqueous carrier; and
c. a perfume;
wherein the deodorizing composition is non-irritating and non-toxic to pets and
wherein the deodorizing composition is adapted to be used with a dirt lifter
comprising a front side comprising textured portions wherein the dirt lifter is adapted to distribute the deodorizing composition through a pet's coat.

10. The deodorizing composition of claim 9 wherein the dirt lifter is further adapted to attract one or more odor causing agents.

11. The deodorizing composition of claim 9 wherein the deodorizing composition further comprises a buffer system comprising citric acid wherein the pH of the deodorizing composition is from about 5 to about 8.5.

12. A method of entrapping and neutralizing malodors on a pet comprising:
a. applying a deodorizing composition on a pet wherein the deodorizing composition comprises a cyclodextrin, an aqueous carrier, and a perfume;
b. distributing the deodorizing composition across at least a portion of the pet's hair using a dirt lifting glove comprising a front side with textured portions and a back side wherein the front side is joined to at least a portion of the back side along the perimeter;
wherein the deodorizing composition is non-toxic and non-irritating to a pet.

13. The method of claim 12 wherein the deodorizing composition further comprises a buffer system and wherein the pH of the deodorizing composition is greater than about 6.

14. The method of claim 13 wherein the buffer system comprises citric acid and sodium citrate.

15. The method of claim 12 wherein the deodorizing composition further comprises a water soluble silicone with an HLB value that is greater than about 10.

16. The method of claim 12 wherein the aqueous carrier further comprises from about 2% to about 7% of a monohydric alcohol.

17. The deodorizing composition of claim 12 wherein the composition is substantially free of a preservative.

18. The method of claim 12 wherein the front side of the dirt lifting glove comprises a facing sheet, an absorbent layer, and an inner sheet where the inner sheet is impermeable to the deodorizing composition.

19. The method of claim 12 wherein the back side comprises a back sheet and wherein the back sheet is extensible.

20. The method of claim 12 wherein at least 3 g of deodorizing composition are used to entrap and neutralize the pet's malodor.
